(19) United States
(12) Patent Application Publication
(10) Pub. No.: US 2022/0204441 A1
(43) Pub. Date: Jun. 30, 2022

(54) OLIGOMERE HEXAFLUOROPROPYLENOXIDE DERIVATIVES

(71) Applicant: Joanneum Research Forschungsgesellschaft mbH, Graz (AT)

(72) Inventors: Dieter Nees, Thannhausen (AT); Stephan Ruttloff, Anger (AT); Johannes Götz, Gleisdorf (AT)

(73) Assignee: Joanneum Research Forschungsgesellschaft mbH, Graz (AT)

(21) Appl. No.: 17/437,183

(22) PCT Filed: Feb. 24, 2020
(86) PCT No.: PCT/EP2020/054739

§ 371 Date: Sep. 8, 2021

(30) Foreign Application Priority Data
Mar. 11, 2019 (DE) 10 2019 106 081.8

(51) Int. Cl. C07C 271/22; C08F 220/34; C09D 133/16; C07C 269/02; G03F 7/00

(52) U.S. Cl. CPC C07C 271/22; C08F 220/346; G03F 7/0002; C07C 269/02; C09D 133/16

(57) ABSTRACT

A compound of formula (I)

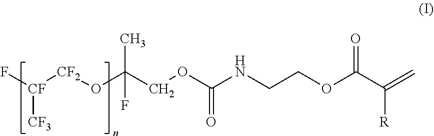

wherein n is selected from 3 to 8 and R is hydrogen or a C₁-C₈ alkyl radical, a process for the preparation of the compound and uses of the compound.

### FIELD OF THE INVENTION

The present invention relates to novel oligomeric compounds based on hexafluoropropylene oxide (HFPO), compositions containing these compounds, substrates whose surfaces have been modified with these compounds, and a process for the preparation and a use of the compounds.

### STATE OF THE ART

In the state of the art, anti-adhesive additives are known which can produce a lotus effect on UV-NIL embossed surfaces (NIL means Nano Imprint Lithography) already in a mass percentage of less than 1% (cf. “The Lowest Surface Free Energy Based on —CF3 Alignment”, Takashi Nishino, Masashi Meguro, Katsuhiko Nakamae, Motonori Matsushita and Yasukiyo Ueda Langmuir 1999, 75, 4321-4323). In addition to the structuring or roughness of the embossed surface, the lotus effect is based on a high proportion of CF₃ groups, which have a much lower surface energy than —CF₂ groups.

A highly surface-active product is the acrylate-functionalized anti-adhesion additive oligo-HFPO-2-hydroxyethyl methacrylate ester (hereinafter also referred to as “HFPO methacrylate”):

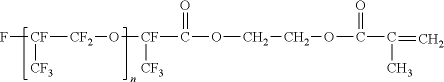

The exceptional surface activity of this molecule is due to the branched structure of the perfluoropolyether chain having the CF₃ side groups. HFPO methacrylate can be prepared by an oxidation of the —CH₂—OH alcohol group of an HFPO alcohol to the carboxylic acid group and subsequent esterification with 2-hydroxyethyl methacrylate.

### Problems to be Solved by the Invention

An object of the present invention is to provide a highly effective anti-adhesion additive and compositions (UV-NIL embossing varnishes) containing the same.

A further object is to provide a simple method of making the non-stick additive and uses thereof.

### SUMMARY OF THE INVENTION

The problem was solved by providing the compound, the composition, the polymer, the coated substrate, the method and the use according to the invention.

These objects according to the invention are defined in the claims.

### Advantages of the Invention

The compound according to the invention has a low surface energy and can be used in a variety of ways as an anti-adhesion additive (e.g. for (meth)acrylate-based UV-NIL embossing varnishes). In this way, the compound according to the invention can significantly reduce the adhesion or demoulding forces during the separation of the embossing stamp from the embossing varnish and also impart a dirt-repellent or self-cleaning property to UV-NIL embossed surfaces and produce a lotus effect on suitable structures.

The compound according to the invention can be prepared in a simpler manner and with higher yield than HFPO methacrylate.

The direct addition of the oligo HFPO alcohols to a 2-isocyanoethyl (meth)acrylate to give the corresponding oligo-urethane (meth)acrylates is simpler and can be carried out in higher yield than the multistep synthesis of HFPO methacrylate.

The starting compounds which can be used for the synthesis of the compound according to the invention are simple and inexpensive to prepare.

### DESCRIPTION OF THE FIGURES

FIG. 1 shows schematically the formation of hydrogen bonds between the urethane groups of compounds according to the invention.

FIG. 2 shows the water and diiodomethane contact angle (KW) on an embossing varnish layer of Example 1, having been UV-cured under inert gas, as a function of the concentration of PFPE-UA-3.

FIG. 3 shows the surface energy on an embossing varnish layer of Example 1, having been UV-cured under inert gas, as a function of the PFPE-UA-3 concentration.

γ: total surface energy; d: disperse fraction, p: polar fraction.

FIG. 4 shows the water and diiodomethane contact angle on an embossing varnish layer of Example 2 as a function of the concentration of PFPE-UA-3.

FIG. 5 shows the surface energy on an embossing varnish layer of Example 2 as a function of PFPE-UA-3 concentration.

γ: total surface energy; d: disperse fraction, p: polar fraction.

### EMBODIMENTS OF THE INVENTION

The present invention provides a compound of formula (I):

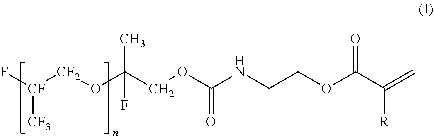

wherein n is selected from 3 to 8 and R is hydrogen or a C₁-C₈ alkyl radical. Preferably, R is hydrogen or the methyl radical.

The polymerizable carbon-carbon double bond can preferably be organically polymerized under the action of light and/or heat and/or by chemical means. It is a group that can be photochemically polymerized under the action of actinic radiation, in particular a UV-polymerizable group.

The polymerisation reaction is usually a polyreaction in which the reactive double bonds or rings are converted into polymers under the influence of heat, light, ionising radiation or chemically (via a redox reaction) (addition polymerisation). The organic polymerization preferably takes place via (meth)acrylic groups.

In addition or alternatively to the polymerization reaction (polyaddition) of the C═C double bonds as such, a reaction of the compounds containing these double bonds with diamines or higher amines or dithiols or higher thiols via a Michael addition (thiol-ene reaction or the analogous reaction with amines, respectively) is also possible.

The compound according to the invention has a high degree of CF₃ residues.

In the structure of formula (I), all CF₃ residues are arranged such that a —CF₂—O— group is interposed in each case.

There is a —CH₂—O group similar to a —CF₂—O group between the urethane group and the adjacent CF₃ residue.

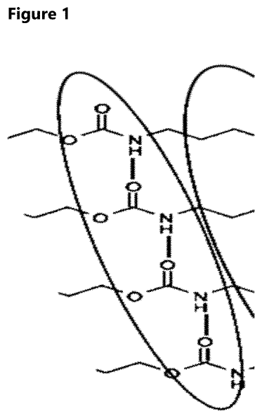

Due to this arrangement, the compound according to the invention has a very regular structure, which enables an ordered and very densely packed fluoro-surfactant-layer structure. This structure is reinforced by the possibility of forming hydrogen bonds between the urethane groups of the compounds according to the invention (cf. FIG. 1).

Overall, this results in the compound according to the invention greatly reducing the surface and thus adhesion energy in embossing varnishes which are, for example, acrylate-based, and thus being able to impart a pronounced lotus effect.

The compound according to the invention is obtainable by a reaction of an alcohol with an isocyanate. The difference to the compound HFPO methacrylate of the prior art arises from the preparation process.

HFPO methacrylate can be prepared by oxidation of the —CH₂—OH alcohol group of an HFPO alcohol to the carboxylic acid group and subsequent esterification with 2-hydroxyethyl methacrylate.

For example, a compound according to the invention is an oligo urethane acrylate and can be prepared, for example, by reacting an oligo HFPO alcohol by addition to 2-isocyanatoethyl acrylate (AOI) to form the urethane acrylate.

This results in the following structural difference between the present invention and HFPO methacrylate:

. . . —CH₂—O—CO—NH—CH₂— . . . Compound of the present invention:

. . . —CO—O—CH₂— . . . HFPO methacrylate:

The oligomer used as the starting compound has units derived exclusively from propylene oxide, so that it can be prepared in a simple manner.

The urethane group present as the head group in the compounds according to the invention is polar due to an additional N atom and can additionally form H-bridge bonds due to the H atom on the N atom. This leads to more highly ordered and more densely packed fluoro-surfactant monolayers on the polymer surface and thus to lower surface energies (cf. FIG. 1).

In the Examples according to the invention, UV-cured embossing varnish layers with different HFPO-UA-3 contents were produced, then the contact angles of water and diiodomethane droplets sitting on these layers were measured and the surface energies were determined from this. The values measured and calculated in this process proved an extraordinary surface activity at least equal to that of HFPO methacrylate. Its surface activity was described in M. Leitgeb, D. Nees et al., ACS Nano 10 (5), 4926 (2016) (therein indicated as PFPE-A1).

The composition according to the invention comprises the compound according to the invention and a polymer starting material. The polymer starting material comprises monomers and/or oligomers having at least one reactive group which can be reacted with the carbon-carbon double bond in the compound according to the invention under polymerization. In the simplest case, this is also a radical having a carbon-carbon double bond. Examples of double bond-containing groups are those having double bonds accessible to Michael addition, such as styryls, norbornenyls or (meth)acrylic acid derivatives; however, they may also be vinyl or allyl groups. By (meth)acrylic derivatives or (meth)acrylic acid derivatives are meant, in particular, the (meth)acrylates and (meth)acrylamides. Additionally or alternatively, the polymer starting material may also comprise residues containing diamines or higher amines or dithiols or higher thiols, which may be reacted via a Michael addition (thiol-ene reaction or the analogous reaction with amines, respectively). In any case, the polymerizable groups of the polymer starting material must be selected to allow polymerization with incorporation of the compound of the invention into the polymer.

The composition according to the invention contains the compound according to the invention in an amount of 0.001 to 10%, preferably 0.001 to 1.0%, and the polymer starting material in an amount of 50 to 99.999%. The remaining components may be, for example, a reactive diluent in a preferred amount of from 5 to 40% and a photoinitiator. Percentages in the present invention are by weight unless otherwise indicated. A preferred composition contains 0.01 to 3% of the compound of the invention, 50 to 80% polymer starting material, 5 to 30% reactive diluent, and 0.1 to 3% photoinitiator, the total amount of these components being at least 90%, preferably at least 95%, of the total amount of the composition of the invention.

A polymer according to the invention is formed after polymerization of the composition according to the invention. The polymer can be in any form, for example in solid form as a film or in liquid form in a facade paint or spray.

The compound according to the invention can be used in the form of the composition according to the invention to coat a substrate. A substrate may be any object, the surface of which is to be provided with the desired anti-adhesion property. Thus, a substrate is, for example, a substrate or support in a microstructure provided with embossing varnish or a stamp for embossing such varnish. However, a substrate can also be any surface, for example a glass surface, the surface of which is to be made dirt-repellent or self-cleaning. Further examples of substrates are surfaces in the field of photovoltaics, lighting or optics, but also textiles, awnings, tarpaulins and sails which are to be made self-cleaning or dirt-repellent.

This coating results in a coated substrate according to the invention having the desired surface properties, in particular an anti-adhesive property and a modified surface energy.

A coated substrate according to the invention may be, for example, a substrate coated with a polymer according to the invention as an embossing varnish, or a working stamp for nano imprint lithography coated with a polymer according to the invention as an anti-adhesion coating, or in which the embossing structure or embossing relief comprises said polymer according to the invention. Such a stamp may comprise a polymer substrate film (e.g. PET) having a structured surface layer comprising the polymer according to the invention at least on its surface. The structured surface layer may be a polymer obtainable by UV curing an acrylate together with the compound according to the invention.

For carrying out a nano imprint lithography process, it may be particularly advantageous if both the stamping varnish and the stamping surface comprise a compound according to the invention.

In the coated substrate according to the invention, the bond between the substrate and the polymer according to the invention can be based in principle on covalent or non-covalent bonds.

Non-covalent bonds may be preferred if, for example, it is desired to allow the layer of the polymer of the invention to be removable. This may be the case, for example, if this layer is to be replaced. An example of such a polymer according to the invention is a thermoplastic.

However, covalent bonds may also be preferred. These can be formed by using appropriate adhesives to bond a polymer of the invention to the substrate. However, they can also be formed by the substrate having bonds with residues that are reacted with the reactive groups of the compound according to the invention during polymerization of the compound according to the invention and are thus incorporated into the polymer, or that are reacted with the reactive groups of the compound according to the invention and/or the other polymerizable components during polymerization of the composition according to the invention and are thus incorporated into the polymer. In this case, the coated substrate according to the invention may be prepared by applying a composition according to the invention to the uncoated substrate and then polymerizing.

The compound according to the invention can be used as an anti-adhesion additive in UV-NIL embossing varnishes and reduce adhesion to working stamps (e.g. made of nickel, quartz or polymers) in the embossing process, and/or it can be used as an anti-adhesion additive in working stamps for UV nano imprint lithography and reduce adhesion to the embossing varnishes in the embossing process.

The anti-adhesive additive can permanently reduce the surface energy of UV-NIL embossed varnish surfaces and thus cause water and dirt repellency and, if necessary, a lotus effect on suitable micro- and nano-structures, i.e. a self-cleaning functionality. The lotus effect is reversibly regenerated by cleaning soiled surfaces with e.g. alcohol.

The compound according to the invention is suitable, among other things, for all UV embossing varnish formulations, for example for UV-NIL embossed surfaces with many different structures (shark skin, moth eyes, diffraction gratings).

Concrete applications are functional surfaces such as anti-reflective coatings (moth-eye effect) as well as dirt-repellent or self-cleaning coatings for photovoltaics, coatings that reduce flow friction (sharkskin effect), lighting, optics, building glazing and the like.

### Preparation of the Compound According to the Invention

In the following, the preparation of the compound according to the invention is described with reference to a preferred embodiment.

The compound according to the invention is obtainable by a reaction of an alcohol with an isocyanate.

Branched CF₃ side group-bearing tri- to hexa-HFPO (oligomer) alcohols are commercially available. Examples thereof include
1H,1H-PERFLUORO-2,5,8-TRIMETHYL-3,6,9-TRIOXADODECAN-1-OL:
2-{1,1,2,3,3,3-hexafluoro-2-[1,1,2,3,3,3-hexafluoro-2-(heptafluoropropoxy)propoxy]propoxy}-2,3,3,3-tetrafluoropropan-1-ol (CAS 14620-81-6)

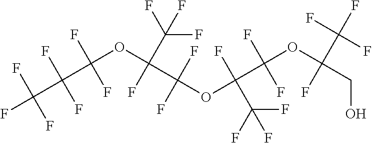 [SMILES: OCC(F)(OC(F)(F)C(F)(OC(F)(F)C(F)(OC(F)(F)C(F)(F)C(F)(F)F)C(F)(F)F)C(F)(F)F)C(F)(F)F]

1H,1H-PERFLUORO(2,5,8,11-TETRAMETHYL-3,6,9,12-TETRAOXAPENTADECAN-1-OL):
2,4,4,5,7,7,8,10,10,11,13,13,14,14,15,15,15-heptadecafluoro-2,5,8,11-tetrakis(trifluoromethyl)-3,6,9,12-tetraoxapentadecan-1-ol (CAS 141977-66-4)

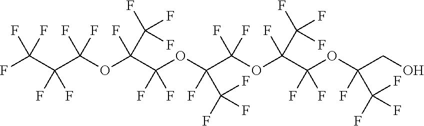 [SMILES: OCC(F)(OC(F)(F)C(F)(OC(F)(F)C(F)(OC(F)(F)C(F)(OC(F)(F)C(F)(F)C(F)(F)F)C(F)(F)F)C(F)(F)F)C(F)(F)F)C(F)(F)F]

1H,1H-PERFLUORO(2,5,8,11,14-PENTAMETHYL-3,6,9,12,15-OXAOCTADECAN-1-OL)(CAS 27617-34-1)

The polyols designated as CAS 14620-81-6 and CAS 141977-66-4 are preferred.

These compounds are linked with 2-isocyanatoethyl acrylate (H₂C═CH—CO—O—CH₂—CH₂—N═C═O; CAS 13641-96-8) to give the corresponding oligo-HFPO urethane acrylates in excellent yields.

A preferred example of such a compound according to the invention is the oligo-HFPO urethane acrylate of formula (I), wherein R may be H and n may be 3 or 4.

### EXAMPLES

The present invention is further illustrated with reference to the following examples.

### Example 1

An embossed varnish layer (75% E8402, 23% nOA, 2% TPO-L) was cured under N₂ inert gas. Various concentrations of PFPE-UA-3 were used as an anti-adhesion additive. The compound PFPE-UA-3 is a compound of formula (I) according to the invention, wherein n is 3 and R is H. E8402 (Ebecryl 8402) is an aliphatic urethane acrylate from Allnex used as an embossing varnish base. The reactive thinner used is n-octyl acrylate (nOA). TPO-L is the photoinitiator ethyl (2,4,6-trimethylbenzoyl)phenylphosphinate.

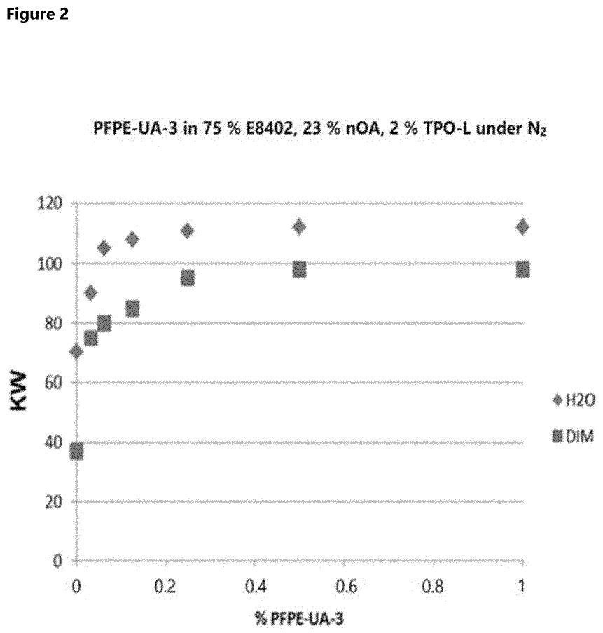

FIG. 2 shows that the contact angles of water and diiodomethane are significantly increased at very low concentrations of PFPE-UA-3 of less than 1%.

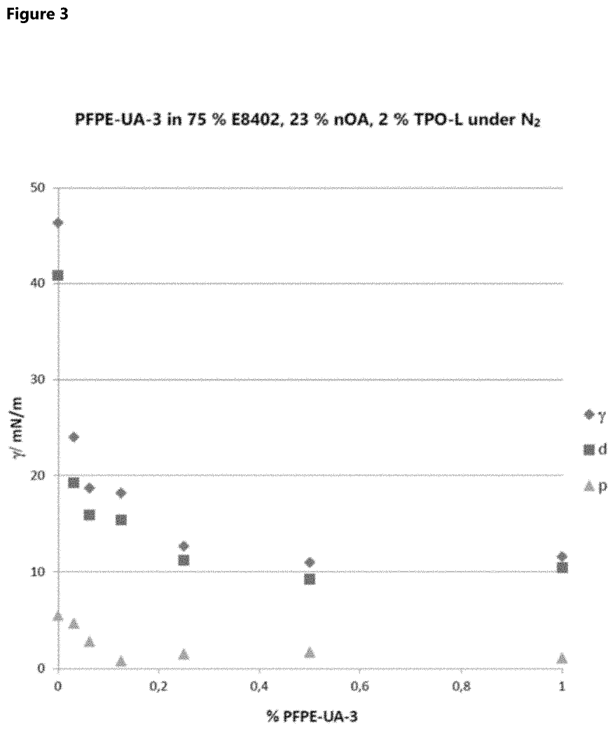

FIG. 3 shows that the surface energy decreased significantly even at low concentrations of PFPE-UA-3, which is mainly due to the decrease of the dispersive part of the surface energy.

In the present invention, the surface energies are determined by the method of Owens, Wendt, Rebel and Kaelble (OWRK) (D. H. Kaelble, Dispersion-Polar Surface Tension Properties of Organic Solids. In: J. Adhesion 2 (1970), pp. 66-81; D. Owens; R. Wendt, Estimation of the Surface Free Energy of Polymers. In: J. Appl. Polym. Sci 13 (1969), pp. 1741-1747; W. Rebel, Einige Aspekte der Benetzungstheorie und ihre Anwendung auf die Untersuchung und Veranderung der Oberflacheneigenschaften von Polymeren. In: Farbe und Lack 77,10 (1971), pp. 997-1005). The OWRK method is a standard method for calculating the surface free energy of a solid from the contact angle with several liquids. The surface free energy is split into a polar fraction and a disperse fraction.

### Example 2

An embossed varnish layer (75% E8402, 23% nOA, 2% TPO-L) was cured against an FPS-coated nickel sheet. Various concentrations of PFPE-UA-3 were used as an anti-adhesion additive.

FPS is 1H,1H,2H,2H perfluorooctylphosphonic acid:

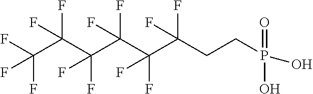 [SMILES: O=P(O)(O)CCC(F)(F)C(F)(F)C(F)(F)C(F)(F)C(F)(F)C(F)(F)F]

This compound forms self-assembled monolayers (SAM) on nickel (FPS-SAM-Nickel) and is used for the anti-adhesion coating of nickel stamps.

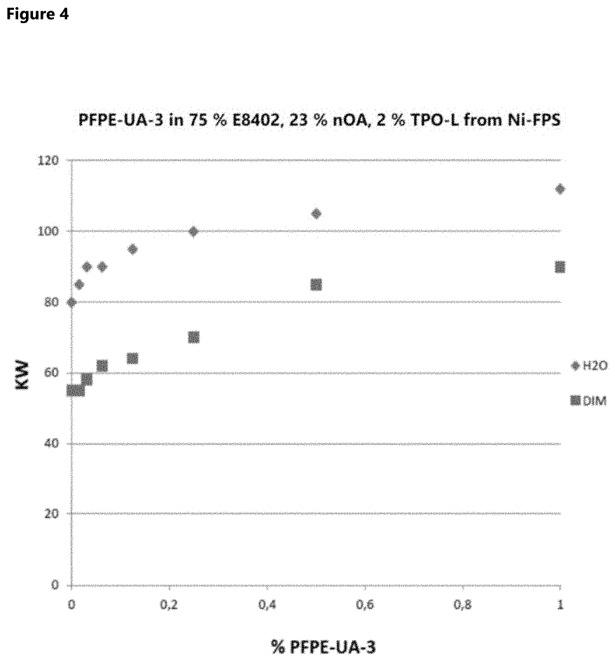

FIG. 4 shows that the contact angle is significantly increased at very low concentrations of PFPE-UA-3 of less than 1%.

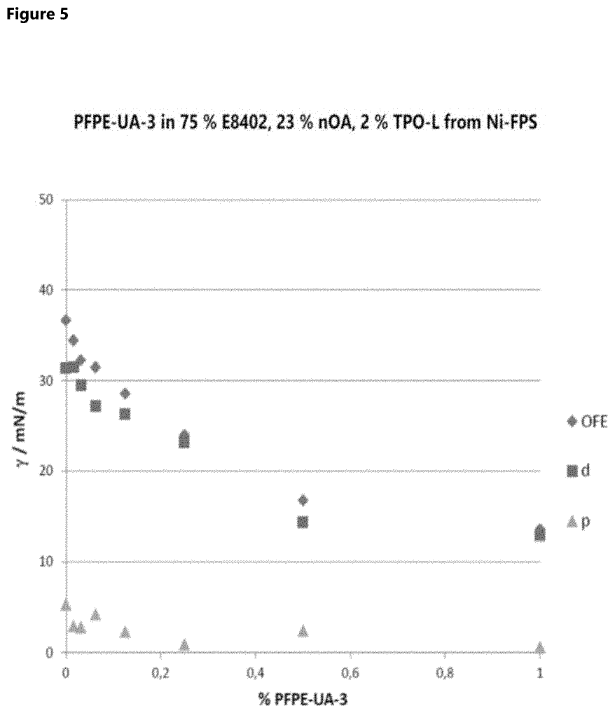

FIG. 5 shows that the surface energy decreased significantly even at low concentrations of PFPE-UA-3.

1. A compound of formula (I) 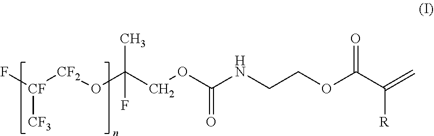
wherein n is selected from 3 to 8 and R is hydrogen or a C₁-C₈ alkyl radical.

2. The compound of claim 1, wherein n is 3 or 4 and R is hydrogen or methyl.

3. A composition comprising the compound of claim 1 and a polymer starting material reactive with the polymerizable carbon-carbon double bond.

4. The composition of claim 3, comprising the compound in an amount of from 0.001 to 10 weight percent and the polymer starting material in an amount of from 50 to 99.999 weight percent.

5. The composition of claim 4, comprising the compound in an amount of from 0.001 to 1.0 weight percent.

6. A polymer obtainable by subjecting the composition according to claim 3 to a polymerization reaction.

7. A coated substrate whose surface comprises a layer containing the polymer according to claim 6.

8. The coated substrate of claim 7, which is a support having disposed thereon an embossed coating layer comprising the polymer obtainable by subjecting the composition to a polymerization reaction, or a stamp for nano imprint lithography having disposed thereon a structured surface layer comprising said polymer.

9. A process for preparing a compound according to claim 1, which comprises preparing the urethane compound in the compound of formula (I) from the corresponding alcohol and the corresponding isocyanate.

10. A process for preparing a polymer comprising polymerizing a polymer starting material in the presence of a compound according to claim 1 whose polymerizable carbon-carbon double bond participates in the polymerization reaction.

11. A nano imprint lithography process which comprises embossing a varnish layer with a stamp, wherein the varnish layer is a composition according to claim 3 and the stamp has a structured surface layer comprising the polymer obtainable by subjecting the composition to a polymerization reaction.

12. The use of a compound according to claim 1 for modifying the surface energy of coatings.

13. The use according to claim 12, wherein the coatings are selected from anti-reflective coatings, dirt repellent coatings, self-cleaning coatings or flow friction reducing coatings.

14. The use according to claim 12 in coatings of materials in the field of photovoltaics, lighting, optics or building glazing.

15. The use of a composition according to claim 3 for modifying the surface energy of coatings.
